(19) United States
(12) Patent Application Publication
(10) Pub. No.: US 2025/0114643 A1
(43) Pub. Date: Apr. 10, 2025

(54) BASE FOR A RADIOTHERAPY DEVICE

(71) Applicant: Elekta Limited, Crawley (GB)

(72) Inventors: Jos Peulen, Crawley (GB); Alexander Harmse, Crawley (GB)

(21) Appl. No.: 18/905,191

(22) Filed: Oct. 3, 2024

(30) Foreign Application Priority Data
Oct. 4, 2023 (GB) 2315241.6

(51) Int. Cl. A61N 5/10; F16M 11/10; F16M 11/42

(52) U.S. Cl. CPC A61N 5/1081; F16M 11/10; F16M 11/42

(57) ABSTRACT

A radiotherapy device can comprise a rotatable gantry and a base. The base can include a wheel configured to support the rotatable gantry and rotate about a wheel rotation axis. The wheel includes an outer surface with first annular edge, a second annular edge, and a central region between the first annular edge and the second annular edge. At least the central region contacts the rotatable gantry's outer surface, rotationally coupling the wheel and gantry. As the wheel's outer surface extends from the central region toward either annular edge, the outer surface of the wheel curves radially inward and away from the gantry's outer surface.

### CLAIM FOR PRIORITY

This application claims the benefit of priority of British Application Serial No. 2315241.6, filed Oct. 4, 2023, which is hereby incorporated by reference in its entirety.

### TECHNICAL FIELD

This disclosure relates to a base for a radiotherapy device, and to a radiotherapy device comprising such a base.

### BACKGROUND

Radiotherapy can be described as the use of ionising radiation, such as X-rays, to treat a human or animal body. Radiotherapy is commonly used to treat tumours within the body of a patient or subject. In such treatments, ionising radiation is used to irradiate, and thus destroy or damage, cells which form part of the tumour.

A radiotherapy device can comprise a gantry which supports a beam generation system, or other source of radiation, which is rotatable around a patient. For example, for a linear accelerator (linac) device, the beam generation system may comprise a source of radio frequency energy, a source of electrons, an accelerating waveguide, beam shaping apparatus, etc.

### SUMMARY

One way to effect rotation of a gantry of a radiotherapy device is to provide a base comprising rotatable drive wheels, and optionally one or more support wheels. The gantry can rest on the wheels of the base in such a way that the wheels and the gantry are rotationally coupled, and rotation of the drive wheels causes rotation of the gantry. This general arrangement is depicted in, for example, FIGS. 2a and 2b of the present application. However, the gantry and all the components supported by it are extremely heavy, and the load on the wheels is not always evenly distributed. Efforts are made to balance the weight of the gantry on the base, however this is a complex undertaking and rotation of the gantry may cause mechanical stresses and strains at the interface between the wheels and the gantry, for example due to mechanical tolerances and other imperfections and deviations. This may cause the gantry to tilt slightly on the base, or else for the weight to be spread unevenly across the wheels of the base. This tilting causes wear or even damage to occur at the interface between the wheels of the base and the gantry. Even in a perfectly balanced system, wear can still occur at this interface due to repeated use.

As radiotherapy devices are developed and improved, there is a demand for increased gantry rotation speed. Increased rotation speed leads to increased patient throughput and reduced treatment times. In addition, radiotherapy devices are typically becoming heavier as their functionality is extended. Increased rotational speed, and the increasing mass of radiotherapy devices, are both likely to further increase wear at the wheel-gantry interface during operation of radiotherapy devices. The present disclosure seeks to address these, and other disadvantages encountered by providing an improved wheel for supporting and driving rotation of a radiotherapy device.

According to an aspect of the present disclosure, there is provided a radiotherapy device comprising a rotatable gantry and a base. The base comprises a wheel configured to support the gantry, and the wheel is configured to rotate about a wheel rotation axis. The wheel comprises an outer surface, the outer surface comprising a first annular edge and a second annular edge, and a central region positioned between the first and second annular edges. At least the central region of the outer surface of the wheel is in contact with an outer surface of the gantry such that the wheel and gantry are rotationally coupled. As the outer surface of the wheel extends from the central region toward at least one of the first and second annular edges, it curves radially inward and away from the gantry outer surface.

According to another aspect, a base for the radiotherapy device is provided. The base comprises a wheel configured to support the gantry. The wheel is configured to rotate about a wheel rotation axis. The wheel comprises an outer surface, the outer surface comprising a first annular edge and a second annular edge, and a central region positioned between the first and second annular edges. At least the central region of the outer surface of the wheel is configured to be in contact with an outer surface of the gantry such that the wheel and gantry may be rotationally coupled. As the outer surface of the wheel extends from the central region toward at least one of the first and second annular edges, it curves radially inward and away from the gantry outer surface.

### FIGURES

Specific embodiments are now described, by way of example only, with reference to the drawings, in which:

FIG. 1 depicts a radiotherapy device;

FIG. 2 depicts a radiotherapy device;

FIG. 3 depicts a radiotherapy device;

FIG. 4 depicts a cross-section through a gantry-wheel interface in accordance with the present disclosure;

FIG. 5 depicts a cross-section through a gantry-wheel interface in accordance with the present disclosure;

FIG. 6 depicts a cross-section through a gantry-wheel interface in accordance with the present disclosure.

### DETAILED DESCRIPTION

In overview, and without limitation, the application discloses a base comprising one or more wheels for supporting a gantry of a radiotherapy device. When viewed in profile, the wheels have an outer circumferential edge which is not flat (as in prior wheel designs), but curved. In other words, the outer surface of each wheel is curved along its width. Each wheel has an annular central region positioned between the two annular outer edges of the wheel. As the outer circumferential surface of each wheel extends from this central region toward at least one of its annular edges, it curves radially inward. In use, wheels having a curved profile as described herein suffer significantly reduced wear, particularly as the gantry of the radiotherapy device tilts slightly with respect to the base.

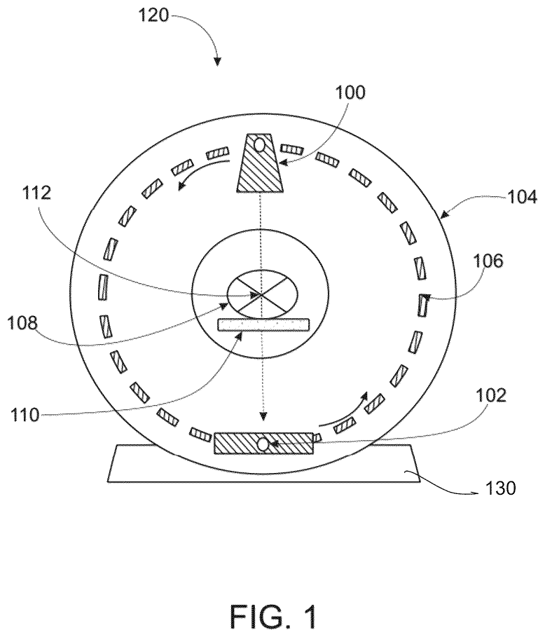

FIG. 1 is a simplified schematic of a radiotherapy apparatus 120. The arrangement should be considered as providing merely an example of a radiotherapy apparatus or the purpose of providing context to the present disclosure, and it will be understood that other arrangements are possible. FIG. 1 depicts a cross-section through the radiotherapy apparatus 120. The apparatus 120 comprises a radiation source 100 and a detector 102 attached to a rotatable gantry 104. The gantry 104 may take the form of a substantially cylindrical drum. The gantry 104 is supported by a base 130 and is rotatable with respect to the base 130. The base comprises a plurality of supporting wheels, including one or more drive wheels (not shown) which are configured to drive rotation of the gantry 104. The radiation source 100 and the detector 102 are fixed to the gantry drum 104 so as to rotate with the gantry drum 104. The radiation source 100 and the detector 102 are arranged diametrically opposite one another.

FIG. 1 also depicts a subject 108 on a support surface 110. The support surface 110 may be moved longitudinally relative to the gantry drum 104 (i.e. away from the plane of the gantry drum 104), for example to aid positioning of the subject 108. In some examples, the support surface 110 may be moved along other translational axes (e.g. in the plane of the gantry) and/or rotational axes. A controller may have access to position and/or movement information for the support surface 110, and may be configured to position a patient according to the requirements of a particular radiotherapy treatment plan. As radiation is applied to the subject 108, for example according to a treatment plan, the radiation source 100 and the detector 102 rotate together with the gantry 104 such that they are always arranged 180° from one another. The radiation source 100 directs radiation towards the subject 108 from various angles around the subject 108 in order to spread out the radiation dose received by healthy tissue to a larger region while building up a prescribed dose of radiation at a target region. As shown in FIG. 1, radiation may be emitted in a plane which is substantially perpendicular to the axis of rotation of the radiation source 100 and gantry 104. Thus, radiation may be applied to a radiation isocentre 112 at the centre of the gantry drum 104 regardless of the angle to which the radiation source 100 is rotated around the gantry drum 104. The radiation can be shaped or limited using a collimator, for example a multi-leaf collimator, arranged in the path of a radiation beam.

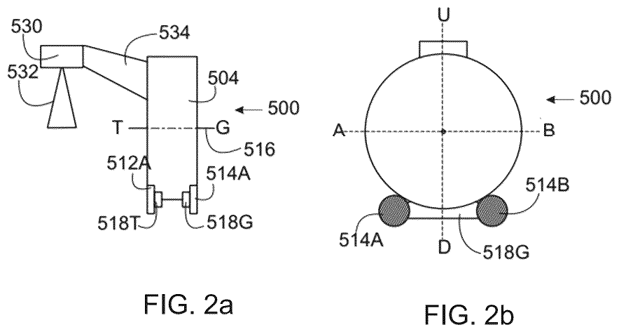

FIGS. 2a-b depict a radiotherapy apparatus 500 which is similar in form, shape and structure to the apparatus 120 depicted in FIG. 1. FIG. 2a depicts the radiotherapy apparatus 500 form the side, and FIG. 2b depicts the radiotherapy apparatus 500 from the rear.

The apparatus 500 comprises a gantry 504 in the form of a drum. The gantry 504 comprises an axis of rotation 516. The apparatus 500 comprises a source of radiation 530 which is coupled to the gantry 504 via an arm 534. FIG. 2a schematically depicts a beam of radiation 532 being emitted from the source of radiation 530. The source of radiation 530 is part of a beam generation system which comprises an electron gun, a waveguide to accelerate electrons emitted by the electron gun toward a target, and a target which, when struck by high energy electrons, produces high energy X-rays.

The gantry rotation axis 516 lies along a T-G axis, where the G refers to the (electron) gun end of the waveguide and the T refers to the target end of the waveguide in an implementation in which the waveguide is comprised within the arm 534. The use of this terminology for the T-G axis is merely used to describe the geometry of the radiotherapy apparatus 500, and does not imply that the waveguide need be oriented in this way in implementations of the present disclosure, or that the apparatus need comprise an electron gun or target arranged in this orientation. The T-G axis passes through the isocenter and is horizontal when the apparatus is viewed from the view depicted in FIG. 2a. The ‘front’ of the device 500, i.e. the side on which the source of radiation 530 is positioned, can be described as the T end of the device 500. Similarly, the rear of the device 500 can be described as the G end of the device 500. The geometry of the device 500 can be further described with respect to an A-B axis, which is depicted in FIG. 2b. The A-B passes through the isocenter, is orthogonal with the T-G axis, and is horizontal when the apparatus is viewed from the view depicted in FIG. 2b. Finally, the apparatus can be described with respect to a U-D (up-down) axis. The U-D axis also passes through the isocenter and is orthogonal with the T-G and A-B axes, and is vertical when viewed from the viewpoint depicted in either FIG. 2a or 2b. Where appropriate for improved clarity of description, certain components herein may be referred to using these axes, and certain reference numerals herein are appended with letters G, T, A, B, U, D, particularly where the component is part of a plurality of similar components and it is useful to distinguish between the components by reference to the apparatus geometry.

The gantry 504 is supported on a plurality of rotatable wheels 512A,B and 514 A,B. Each of wheels 512A,B, is positioned toward the front of the apparatus (i.e. at the “T” end of the apparatus 500), and each of wheels 514A,B is positioned toward the rear of the apparatus 500 (i.e. at the “G” end of the apparatus 500). The wheels are substantially cylindrical, and may take the form of a tradition wheel shape or else elongated cylinders. Herein, the term “rollers” may also be used to describe the wheels. A wheel, or roller, of the base may simply be a rotatable component on which the gantry is supported.

Each of the wheels 512A,B and 514 A,B is positioned underneath the gantry 504 and is configured to support the gantry 504. The wheels 512A,B and 514 A,B are each in contact with the gantry 504. At least part of the outer circumferences, i.e. the radially outermost surfaces, of the wheels 512A,B and 514 A,B are in contact with an outer circumference of the gantry 504. The wheels 512A,B and 514 A,B are rotationally coupled with the gantry 504 such that the wheels 512A,B and 514 A,B and the gantry 504 rotate together about their respective rotational axes.

One or more of the plurality of wheels 512A,B and 514 A,B may be a drive wheel. In some implementations, all of the wheels 512A,B and 514 A,B are drive wheels. The one or more drive wheels are coupled with a motor or other drive means (not depicted). The one or more drive wheels enable rotation of the gantry 504 by actuation of the motor or other drive means. To rotate the gantry, the motor(s) or other drive means control rotation of the drive wheels, which via friction causes the gantry 504 to rotate. As each of the wheels 512A,B and 514 A,B is rotationally coupled with the gantry 504, each of the wheels rotates with the gantry 504 regardless of whether or not it is a drive roller.

In the implementation shown in FIGS. 2a, 2b, there are four rotatable rollers, or wheels, configured and positioned to support the gantry 104 However it should be understood that in some implementations there may be more, or fewer. In the implementation shown, the four wheels comprise a plurality of ‘front’ drive wheels 512A,B. These front wheels are at the T end of the gantry 504. The plurality of drive wheels further comprises a plurality of ‘rear’ drive wheels 514A,B. These rear wheels 514A,B are at the G end of the gantry 504. In other words, FIGS. 2a,b depict an implementation in which there are four drive wheels total, with two front drive wheels 512A,B and two rear drive wheels 514A,B. By positioning two wheels toward the front end of the gantry and two wheels toward the rear end of the gantry, the gantry is well-supported.

The radiotherapy apparatus 500 also comprises a base comprising support structure for supporting the wheels 512A,B and 514 A,B. The support structure(s) supports the rotatable wheels 512A,B and 514 A,B and holds them in place as they rotate. In FIGS. 2a,b the base comprises two support structures: a front support structure 518T toward the T end of the apparatus 500 which supports the front wheels 512A and 512B; and a rear support structure 518G toward the G end of the apparatus 500 which supports the rear wheels 514A and 514B. However, it will be appreciated that the base may comprise a single support structure which supports both the rear and the front wheels. The support structures 518T,G may support the wheels, for example, via axles and axle holders (not shown in FIGS. 2a, 2b).

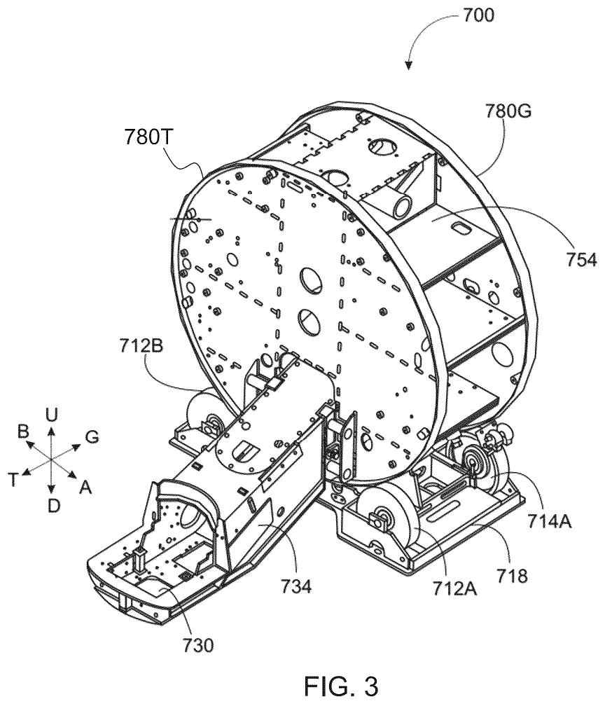

FIG. 3 depicts a radiotherapy apparatus 700 according to the present disclosure having a plurality of support wheels, including two front wheels 712A and 712B and two rear wheels comprising an A-side rear wheel 714A and a B-side rear wheel 714B (not shown in the figure). A mechanical arm 534 extends from the gantry 754 toward housing 730. While not shown here, the housing 730 is configured to hold the radiation source and/or the beam generation system.

The front wheels 712A,B are configured to rotate about their respective axles, which are each held in place by respective axle holders on the base 718. The base 718 may take the form of an under-gantry support structure. The front wheels 712A,B are displaced from one another along the A-B axis, and the rear wheels 712A,B are also displaced from one another along the A-B axis. The front wheels 712A,B are displaced from the rear wheels 714A,B along the T-G axis. Any single wheel or combination of the front or rear wheels may be drive wheels, however in this implementation each of the front and rear wheels is a drive wheel (drive means such as rotary motors not shown in the figure). The gantry further comprises two rims 780 around its circumference. These rims 780 are separated along the gantry rotation axis, and equivalently along the T-G axis. Each rim 780 is a radial outer surface of the gantry. The rims are substantially annular in shape. The front wheels 712A,B support the gantry 754 via contact with the front rim 780T, and where the rear wheels 714A,B support the gantry 754 via contact with the rear rim 780G. By driving each of the wheels together, the gantry may be rotated, and the angle of rotation of the gantry 754 (and hence the radiation source) can be controlled.

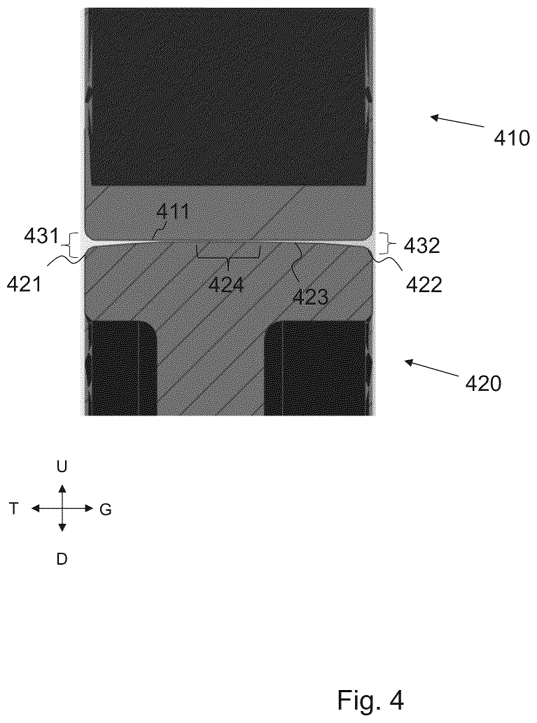

FIG. 4 depicts a schematic of an interface between a rim 410 of a gantry of a radiotherapy device and a wheel 420 according to the present disclosure. The interface is shown in cross-section, and side-on. In other words, FIG. 4 depicts a cross-section through the wheel 420 in a plane which contains the wheel axis of rotation, and which is parallel to both the U-D and T-G axes. The gantry and radiotherapy device may be in accordance with the device 700 depicted in FIG. 3. In particular, the wheel 420 may be in accordance with any of the wheels 712A,B, 714A,B depicted in FIG. 3. The rim 410 may be in accordance with either the front rim 780T or back rim 780G depicted in FIG. 3.

In an example implementation, the wheel 420 may be a drive wheel coupled to a motor (not shown) and configured to not only support the gantry but also drive rotation of the gantry. Alternatively, the wheel 420 may be a support wheel configured to support the gantry as it rotates, but not configured to drive rotation.

The wheel 420 is configured to rotate about a wheel rotation axis. This rotation axis is parallel to the T-G axis. The wheel rotation axis is substantially parallel with the gantry rotation axis. In optimal use, the gantry rotation axis may be perfectly parallel with the wheel rotation axis. However, the gantry is extremely heavy and has an uneven distribution of mass. Therefore, in use, the gantry may tilt slightly such that the gantry rotation axis is not entirely parallel, in a strict mathematical sense, with the wheel rotation axis. However, this tilting is typically less than 0.1°, is not noticeable to the average observer, falls within clinical and regulatory guidelines, and therefore falls within the definition of “substantially parallel” as used herein. This tilting of the gantry causes stresses and strains to occur at the wheel-gantry interface depicted in FIG. 4, and is a major contributor to wear of the wheel 420.

The wheel 420 comprises an outer surface 423. The outer surface 423 is the radially outermost surface of the wheel 420, and may be described as a circumferential surface. The outer surface 423 of the wheel is configured to contact an outer surface 411 of the gantry. In particular, the outer surface 423 of the wheel is configured to contact an outer surface 411 of a rim 410 of the gantry.

The outer surface of the wheel 423 comprises a first edge 421, and a second edge 422. The outer surface 423 extends between the first edge 421 and the second edge 422. As can be appreciated from the figure, the first edge 421 is separated from the second edge 422 in a direction parallel with the wheel rotation axis (or, equivalently, parallel with the T-G axis). The first and second edges 421, 422, are both annular in shape. The first and second edges 421, 422 define the extent of the outer surface 423 in a direction parallel to the wheel rotation axis.

The outer surface 423 of the wheel 420 also comprises a central region 424. The central region 424 is positioned along the outer surface 423, between the first edge 421 and the second edge 422. As can be appreciated from FIG. 4, at least the central region 424 of the outer surface 423 of the wheel 420 is in contact with the outer surface 411 of the gantry. By virtue of this contact, the wheel 420 and gantry are rotationally coupled, such that the wheel 420 and gantry can rotate together. In an implementation in which the wheel 420 is a drive wheel, this contact and rotational coupling enables the wheel 420 to drive rotation of the gantry.

As can be appreciated from FIG. 4, the outer surface 423 of the wheel 420 is curved, not only around its circumference but also in cross-section. This is in contrast to prior wheels used to support radiotherapy devices, which to date have had flat, square cross-sections which are not curved in this manner. The outer surface 423 is curved in such a manner that, in optimal operation of the radiotherapy device, when the gantry rotation axis is exactly or almost exactly parallel with the wheel rotation axis for example, a first small gap 431 is formed between the first edge 421 and the outer surface 411 of the gantry, and a second small gap 432 is formed between the second edge 422 and the outer surface 411 of the gantry.

The creation of a small gap on at least one side of the wheel in this manner, and preferably on both sides of the wheel, allows room for the gantry to tilt slightly without causing wear or damage to the wheel 420. As the gantry tilts, a contact region between the outer surface 423 of the wheel 420 and the outer surface of the gantry may change location and/or shape. However, crucially, the contact region can change location and/or shape without causing significant additional wear to the surface of the wheel, or the outer surface of the gantry (e.g. the rim 410 of the gantry).

In more detail, and as can be appreciated from FIG. 4, as the outer surface 423 of the wheel extends from the central region 424 toward the first annular edge 421, it curves radially inward. The outer surface 423 curves toward the rotation axis of the wheel 420 as it extends toward the first edge 421. In so doing, the outer surface 423 also curves away from the outer surface 411 of the gantry. This curvature creates the first gap 431. In one implementation of the present disclosure, the wheel 420 may be curved in this manner on just one of its sides, such that tilting of the gantry in a corresponding direction does not increase wear on the wheel.

However, in a preferred implementation, the outer surface 423 is curved on either side of the central region 423. In this implementation, the outer surface 423 of the wheel curves radially inward as it extends from the central region 424 toward the second annular edge 422 too. In other words, the outer surface 423 curves toward the rotation axis of the wheel 420 as it extends toward both the first edge 421 and the second edge 421. In so doing, the outer surface 423 also curves away from the outer surface 411 of the gantry on either side of the central region 424. This curvature creates both the first gap 431 and the second gap 432.

As the outer surface 423 extends from the central region 424 toward the first annular edge 421, it curves radially inward and away from the gantry outer surface to define a first curved region, and as the outer surface 423 extends from the central region 424 toward the second annular edge 422, it curves radially inward and away from the gantry outer surface to define a second curved region. As depicted in FIG. 4, the central region 424 may also be curved, and in an implementation the outer surface 424 comprises a smooth, continuous curve across its entire width as depicted in FIG. 4.

The wheel 420 has a width measured parallel to the wheel rotation axis. The width of the central region 424 has a width which is less than the full width of the wheel 420. A radial distance between the wheel rotation axis and the outer surface 423 of the wheel 420 varies across the width of the wheel 420. By virtue of the curvature of the outer surface 423 described above, this radial distance decreases from the central region 424 toward each of the first edge 421 and second edge 422. This means that a radius of the wheel 420 at the central region 424 is greater than both a first radius measured at the first end 421 and a second radius measured at the second end 422. This is in contrast with prior designs, in which this radial distance remains constant across the width of the wheel.

In an implementation in which the central region 424 is curved such that the outer surface 423 is curved across its width, as depicted in FIG. 4, then the central region 424 can be thought of as having a central ring, positioned equidistantly from each of the first and second annular edges 421, 422. Points on this central ring of the outer circumferential surface 423 of the wheel 420 are located at the greatest distance from the wheel rotation axis compared to other points positioned on the outer surface 423. Since the cross-sectional diagram of FIG. 4 depicts the interface in two-dimensions and doesn't depict the entire cross-section of the wheel 420, only a single point on this central ring can be seen in FIG. 4. This single visible point of the central ring lies at the centre of central region 424. As the outer surface 423 of the wheel 420 extends from this central ring toward both the first and second annular edges 421, 422, the outer surface 423 curves radially inward and away from the gantry outer surface.

Wheels of the form depicted in FIG. 4 may be used in the base 718 depicted in FIG. 3. Such a base 718 may comprise a single one of the wheels described above (and elsewhere herein) or may comprise a plurality of such wheels. For example, each of the front wheels 712A,B and rear wheels 714A,B may take the form described above. In such an example, a subset of the wheels are drive wheels, and a subset of the wheels are supporting wheels configured to support the gantry but not drive rotation.

The base 718, therefore, may comprise a plurality of wheels, each with an outer surface 423 comprising first and second annular edges 421,422 and a central region 424 positioned therebetween. For each of the wheels, at least the central region is in contact with an outer surface of the gantry. For example, the front wheels 712A,B may be configured and positioned in the base to support and contact a first, front rim 780T of the gantry, and the rear wheels 714A,B may be configured and positioned in the base to support and contact a second, rear rim 780T of the gantry. In this way, or in any other manner known to the skilled person, the wheels of the base 718 are rotationally coupled to the gantry. As described above in relation to only a single wheel, each of the wheels of the base 718 has an outer surface which curves radially inward and away from the gantry outer surface as it extends from its central region toward at least one of its first and second annular edges. When the gantry of the radiotherapy device is supported on such a base, it can tilt with respect to the rotational axes of each of the wheels, and even move or ‘walk’ slightly with respect to the base, without causing additional wear to the wheels.

The wheel(s) disclosed herein are advantageous. The curved profile of the wheels allows the gantry to tilt slightly in normal use of the radiotherapy device without causing significant stress or wear on the wheels. This reduces the number of times the wheels need to be repaired or replaced in a service or repair action. The design increases efficiency, in the sense that the gantry can be rotated more times for a given degree of wear at the wheel-gantry interface. The radiotherapy device may therefore remain in operation for longer periods of time, or at least can be repaired and serviced more efficiently. In turn, patient throughput of the radiotherapy device can be increased.

By virtue of the outer surfaces of the wheels curving radially inward and away from the gantry outer surface in the manner described above, small gaps 431, 432 may be created between the outer surface of the wheel(s) and the outer surface(s) of the gantry. These gaps allow for a small degree of tilt of the gantry. It should be understood that, given the gantry is incredibly heavy, the weight of the gantry may distort the outer surface of the wheels such that there is no visible gap between wheel and gantry in use, however due to the shape of the wheel the contact pressure between the wheel and gantry is nevertheless lessened. Regardless of the presence of gaps 431, 432 which are visible to the naked eye in use, the curved profile of the wheel(s) allows for a degree of tilting and movement of the gantry relative to the base and wheels. This contrasts significantly with existing designs, in which the wheels of the base are not designed to allow for this tilting or movement. Existing methods of preventing wear at the gantry-wheel interface have focused on reducing the degree of tilt of the gantry using, for example, load balancing techniques. These techniques can be effective, but are complex and expensive. Alternative techniques have focused on material selection, but high-performance materials in terms of reduced wear are expensive and have downsides in other performance areas. An additional advantage of the solutions set out in the present disclosure, compared to prior techniques, is that the presently disclosed wheels are straightforward and inexpensive to manufacture.

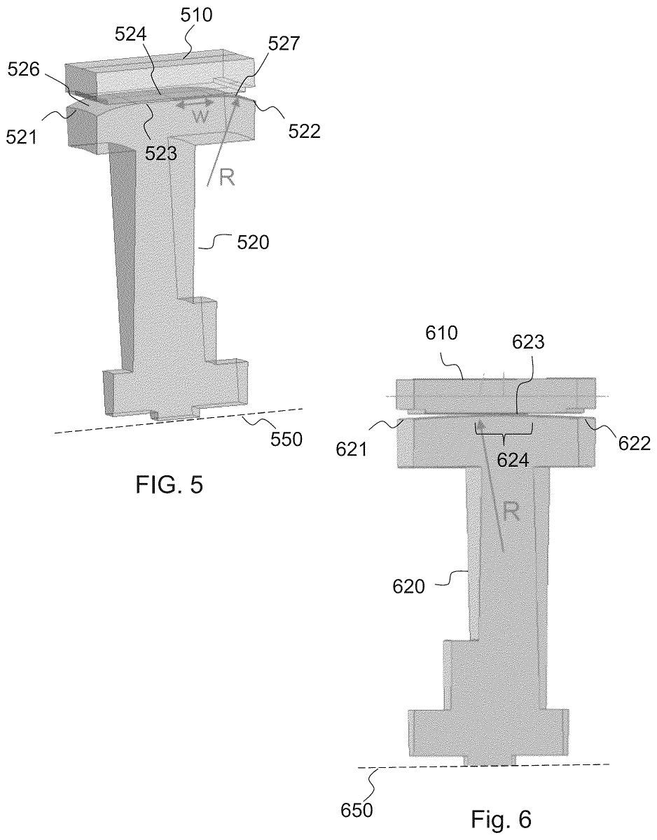

FIGS. 5 and 6 depict specific implementations of the general concepts explained above with respect to FIG. 4. Factors such as the degree of curvature of the outer surface, the extent and shape of the central region, depend on the load case and operational parameters. FIG. 5 depicts a first embodiment which testing has shown to be optimal where gantry tilts are expected to be small, e.g. <0.01°. FIG. 6 depicts a second embodiment which testing has shown to be optimal where gantry tilts are expected to be slightly greater, e.g. up to 0.1°.

FIG. 5 depicts a cross-section through an interface between wheel 520 and an outer surface (such as an outer rim) of a gantry 510. Only half of the wheel cross-section is shown in FIG. 5. Both components are shown at an angle and with a degree of transparency to enable greater understanding.

To enable quick understanding of the figure, the rough position of the wheel rotation axis 550 is depicted by a dashed line. In an implementation, this rotation axis 550 is co-aligned with a central axis of the wheel.

As can be appreciated from FIG. 5, the wheel 500 comprises an outer surface 523. The outer surface 523 is generally annular in shape and may be described as a circumferential outer surface or a radially outermost surface. The outer surface 523 comprises a central region 524. Only a portion of the central region 524 is depicted here, as it will be appreciated that the central region 524 is substantially annular and extends around a circumference of the wheel 520. The central region 524 also extends in a direction parallel to the rotation axis 550. In cross-section, it can be appreciated that the central region 524 is substantially flat; in other words, is not curved. The radius of the central region 524 is substantially constant along its width, and does not curve radially inward or outward with respect to the wheel rotation axis 550.

As can be appreciated from FIG. 5, the outer surface 523 is curved on either side of the central region 524. As the outer surface 523 of the wheel 520 extends from the central region 524 toward its first annular edge 521, it curves radially inward (toward the rotation axis 550) and away from the gantry outer surface to define a first curved region 526. As the outer surface 523 of the wheel 520 extends from the central region 524 toward its second annular edge 522, it also curves radially inward and away from the gantry outer surface to define a second curved region 527. As with the central region 524, it should be appreciated that only a portion of the curved regions 526, 527 are depicted herein. The full curved regions 526, 527 are substantially annular and each extend around the outer circumference of the wheel 520. Each of the first curved portion 526 and second curved portion 527 flare inwards toward the outer axial edges 521, 522 of the outer surface 523 and may be described as flared regions or flared portions for this reason. From inspection of the figure it can be appreciated that a distance between the outer surface 523 of the wheel and the outer surface of the gantry rim 510 increases as the outer surface 523 extends from each side of the central region toward the annular edges 521, 522, and that as this distance increases the radius of the outer surface 522 measured from the wheel rotation axis 550 increases.

The central region 524 has a width of 2 W (where W is a ‘half-width’, i.e. a distance from a central point or ring of the central region 524 to the start of the first or second curved portion 526, 527). The curved portions 526, 527 have a radius of curvature, R. This radius of curvature R may be constant radius of curvature. In a specific implementation. W may be 5 mm and R may be 100 mm, and the total wheel width is 80 mm. In this implementation, it will be appreciated that, in cross-section as depicted in FIG. 5, approximately 87.5% of the wheel's outer circumference is curved, with approximately 12.5% being flat. During testing, it has been found that a wheel with over 75% of its outer surface being curved in this way brings significant benefits, with improved results for over 80% being curved, and still better results for over 85%.

Turning to the specific implementation depicted in FIG. 6, FIG. 6 depicts part of a wheel 620 in cross-section, with a wheel rotation axis 650 marked with a dashed line to aid understanding. This implementation is similar in form to that depicted in FIG. 4. In this implementation, the outer surface 623 has a constant radius of curvature, R, across its entire width. The outer surface 623 of the wheel 620, when viewed in a cross-sectional plane that contains the wheel rotation axis (as depicted in FIG. 6), is smoothly curved along its entire width. The central region 624 is itself curved and comprises the constant radius of curvature, R. Testing indicates that a radius of curvature of 2000 mm is optimal for use cases in which larger gantry tilt angles might be expected, e.g. up to 0.1°.

In this implementation, the central region 624 can be thought of as comprising a central ring, as described above in relation to FIG. 4. The outer surface 623 of the wheel 620 curves away from this central ring toward both the first and second annular edges 621, 622. The curving away can be described as sloping away, such that the outer surface 623 can be described as sloping away from the central ring. As with the implementations depicted in FIGS. 4 and 5, the outer surface 623 of the wheel 620 extends from the central region 624 toward each of the first and second annular edges 621, 622, and as it does so it curves radially inward (toward the wheel rotation axis 650) and away from the gantry outer surface. This curved profile can be described as a radial bulge, such that the wheel 620 can be described as bulging radially outward at a central region of its axial width.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A radiotherapy device comprising:
a rotatable gantry; and
a base, wherein the base comprises a wheel configured to support the rotatable gantry, wherein the wheel is configured to rotate about a wheel rotation axis, wherein the wheel comprises an outer surface, the outer surface comprising a first annular edge, a second annular edge, and a central region positioned between the first annular edge and the second annular edge, wherein at least the central region of the outer surface of the wheel is in contact with an outer surface of the rotatable gantry such that the wheel and gantry are rotationally coupled, and wherein, as the outer surface of the wheel extends from the central region toward at least one of the first annular edge or the second annular edge, the outer surface of the wheel curves radially inward and away from the outer surface of the rotatable gantry.

2. The radiotherapy device of claim 1, wherein the first annular edge and the second annular edge are separated in a direction parallel to the wheel rotation axis.

3. The radiotherapy device of claim 1, wherein the outer surface of the wheel is a radially outermost surface.

4. The radiotherapy device of claim 1, wherein the outer surface of the wheel is a circumferential surface.

5. The radiotherapy device of claim 1, wherein the wheel has a width measured parallel to the wheel rotation axis, and a radial distance between the wheel rotation axis and the outer surface of the wheel varies across the width of the wheel.

6. The radiotherapy device of claim 5, wherein the radial distance decreases from the central region toward each of the first annular edge and the second annular edge.

7. The radiotherapy device of claim 5, wherein a radius of the wheel at the central region is greater than both a first radius at the first annular edge and a second radius at the second annular edge.

8. The radiotherapy device of any of claim 5, wherein the radial distance varies across the width of the wheel such that the outer surface of the wheel is curved across at least 75% of the width of the wheel.

9. The radiotherapy device of any of claim 5, wherein the outer surface of the wheel has a constant radius of curvature along its width.

10. The radiotherapy device of claim 1, wherein a distance between the outer surface of the wheel and the outer surface of the rotatable gantry increases as the outer surface of the wheel extends from the central region toward at least one of the first annular edge or the second annular edge.

11. The radiotherapy device of claim 1, wherein the wheel has a width measured parallel to the wheel rotation axis, and the outer surface is smoothly curved along its width.

12. The radiotherapy device of claim 1, wherein the outer surface is curved on either side of the central region, such that:
as the outer surface of the wheel extends from the central region toward the first annular edge, the outer surface of the wheel curves radially inward and away from the outer surface of the rotatable gantry to define a first curved region; and
as the outer surface of the wheel extends from the central region toward the second annular edge, the outer surface of the wheel curves radially inward and away from the outer surface of the rotatable gantry to define a second curved region.

13. The radiotherapy device of claim 12, wherein the central region is an annular region which extends around a circumference of the wheel, and in a direction substantially parallel with the wheel rotation axis.

14. The radiotherapy device of claim 12, wherein the central region has a radius which does not vary across its width.

15. The radiotherapy device of any of claim 12, wherein each of the first curved region and the second curved region comprise a radius of curvature of substantially 100 mm.

16. The radiotherapy device of claim 1, wherein the rotatable gantry is configured to rotate about a gantry rotation axis, and wherein the gantry rotation axis is substantially parallel with the wheel rotation axis.

17. The radiotherapy device of claim 1, wherein the wheel is a drive wheel configured to drive rotation of the rotatable gantry.

18. The radiotherapy device of claim 1, wherein the base further comprises a plurality of additional wheels, wherein each wheel of the plurality of additional wheels comprises:
a respective outer surface comprising a respective first annular edge and a respective second annular edge and a respective central region positioned therebetween, wherein at least the respective central region of the outer surface of each wheel in the plurality of additional wheels is in contact with at least a portion of the outer surface of the rotatable gantry such that the rotatable gantry is rotationally coupled to each wheel of the plurality of additional wheels, and wherein, as the respective outer surface of each wheel of the plurality of additional wheels extends from the respective central region toward at least one of the respective first annular edge or the respective second annular edge, the respective outer surface curves radially inward and away from the outer surface of the rotatable gantry.

19. A base for a radiotherapy device comprising a rotatable gantry, the base comprising:
a wheel configured to support the rotatable gantry, wherein the wheel is configured to rotate about a wheel rotation axis, wherein the wheel comprises an outer surface, the outer surface comprising a first annular edge, a second annular edge, and a central region positioned between the first annular edge and the second annular edge, wherein at least the central region of the outer surface of the wheel is configured to be in contact with an outer surface of the rotatable gantry such that the wheel and the rotatable gantry are rotationally coupled, and wherein, as the outer surface of the wheel extends from the central region toward at least one of its first annular edge or the second annular edge, the outer surface curves radially inward and away from the outer surface of the rotatable gantry.

20. The base of claim 19, wherein the wheel has a width measured parallel to the wheel rotation axis, and a radial distance between the wheel rotation axis and the outer surface of the wheel varies across the width of the wheel such that the outer surface of the wheel is curved across at least 75% of the width of the wheel.
